(19) United States
(12) Patent Application Publication
(10) Pub. No.: US 2006/0211123 A1
(43) Pub. Date: Sep. 21, 2006

(54) Sensing element and method of making

(76) Inventors: Eric L. Ker, Grand Blanc, MI (US); Raymond L. Bloink, Swartz Creek, MI (US); Carlos A. Valdes, Flint, MI (US); David K. Chen, Rochester Hills, MI (US); Jinping Zhang, Grand Blanc, MI (US)

Correspondence Address: Jimmy L. Funke;Delphi Technologies, Inc., M/C 480-410-202, P.O. Box 5052, Troy, MI, 48007, US

(21) Appl. No.: 11/083,509

(22) Filed: Mar. 18, 2005

(51) Int. Cl. G01N 33/00

(52) U.S. Cl. 436/113

(57) ABSTRACT

A sensing element and a method of making the same are provided. The sensing portion of the element comprises an inorganic binder and a sensing material. In some instances, the sensing material can be an ammonia sensing material.

### TECHNICAL FIELD

The present disclosure is related to a sensing element responsive to a gas and, in particular, to an ammonia-sensing element that is responsive to ammonia.

### BACKGROUND

Exhaust gas generated by combustion of fossil fuels in furnaces, ovens, and engines, for example, contains nitrogen oxides (NOx), unburned hydrocarbons (HC), and carbon monoxide (CO). Vehicles, e.g., diesel vehicles, utilize various pollution-control after treatment devices such as, for example, a NOx absorber or Selective Catalytic Converter (SCR), to reduce NOx. For diesel vehicles using SCR, the NOx reduction can be accomplished by using ammonia gas (NH₃). In order for SCR catalyst to work efficiently, and to avoid pollution breakthrough, an effective feedback control loop is needed.

To develop such control technology, there is an ongoing need for economically produced and reliable commercial ammonia sensors.

### SUMMARY

The present disclosure is directed, in one embodiment, to a sensing element. The sensing element comprises a heater section comprising a heater and a temperature sensor, and a sensing section comprising an impedance-measuring device and a sensing portion disposed adjacent the impedance-measuring device opposite the heater section. The sensing portion comprises a mixture of a sensing material and an inorganic binder. A first insulating layer is disposed between the heater and the temperature sensor and a second insulating layer is disposed between the temperature sensor and the impedance-measurement device.

Another embodiment of the disclosure is directed to a method of making a sensing element. The method comprises forming a green laminate heater section comprising a first insulating layer, a heater disposed on one side of the first insulating layer and a temperature sensor disposed on the opposite side of the first insulating layer. The green laminate heater section is heated to form a heater section. An impedance-measuring device pattern is formed on the temperature sensor side of the heater section. A sensing material precursor comprising an inorganic binder and a sensing material can be formed and disposed over the impedance-measuring device pattern. The sensing material pre-cursor is heated to form the sensing element comprising a sensing portion.

In some embodiments of the foregoing device and method, the sensing material comprises an ammonia sensing material.

The above described and other features are exemplified by the following figures and detailed description.

### DRAWINGS

Refer now to the figures, which are meant to be exemplary, not limiting, and wherein the like elements are numbered alike.

FIG. 1 is an exploded, isometric view of an exemplary ammonia-sensing element.

FIG. 2 is an isometric view of a portion of the heater section of the exemplary ammonia sensor of FIG. 1, showing the heater.

FIG. 3 is an isometric view of a portion of the sensing section of the exemplary ammonia sensor of FIG. 1, showing the interdigitated capacitor.

FIG. 4 is a cross-sectional view through line 4-4 of FIG. 3.

FIG. 5 is a graphical representation of the durability output for the ammonia-sensing element according to FIG. 1.

FIG. 6 is a graphical representation of the durability output for a comparative ammonia-sensing element.

### DETAILED DESCRIPTION

At the outset of the detailed description, it should be noted that the terms “first,” “second,” and the like herein do not denote any order or importance, but rather are used to distinguish one element from another, and the terms “a” and “an” herein do not denote a limitation of quantity, but rather denote the presence of at least one of the referenced items. Furthermore, all ranges disclosed herein are inclusive and combinable (e.g., ranges of “up to about 25 weight percent (wt. %), with about 5 wt. % to about 20 wt. % desired, and about 10 wt. % to about 15 wt. % more desired,” are inclusive of the endpoints and all intermediate values of the ranges, e.g., “about 5 wt. % to about 25 wt. %, about 5 wt. % to about 15 wt. %”, etc.).

The present disclosure is directed to a sensing element for determining the concentration of a selected gas in a gaseous stream such as a vehicle exhaust gas stream, as well as a method of making such a sensing element.

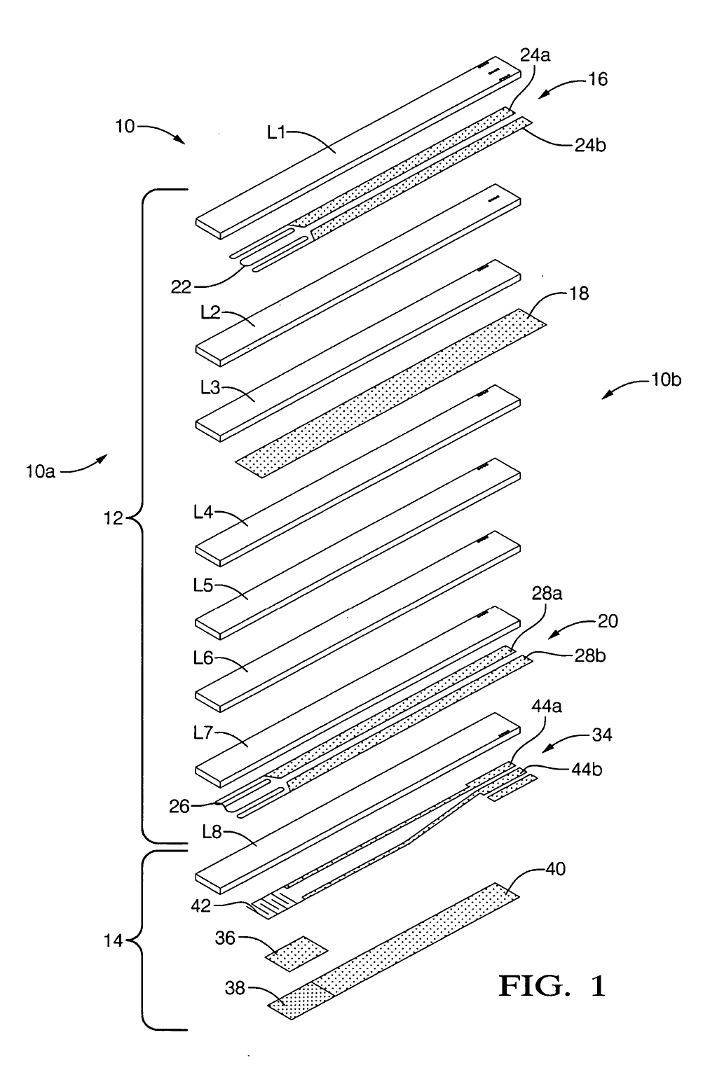

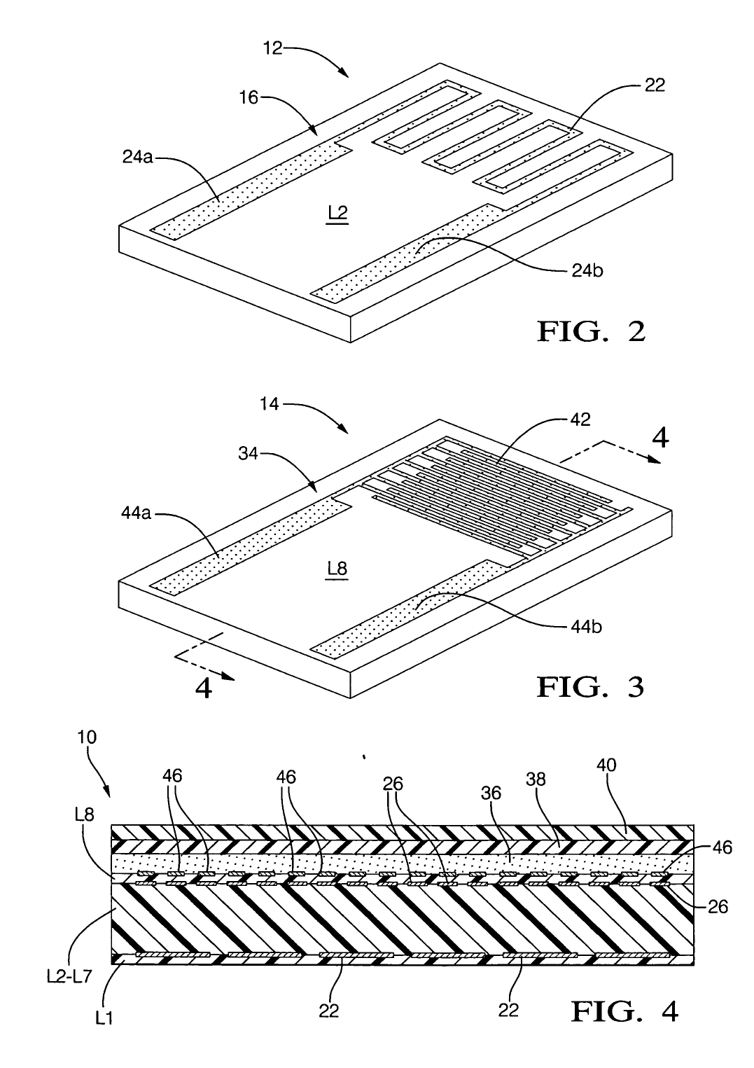

FIGS. 1-4, when taken together, illustrate an exemplary sensing element 10 according to the present disclosure, which is an ammonia-sensing element. Ammonia sensing element 10 comprises a sensing end 10a, a terminal end 10b opposite the sensing end 10a, a heater section 12 and a sensing section 14.

The exemplary sensing element 10 comprises insulating layers L1-L8, but it should be understood that the number of insulating layers could vary depending on a variety of factors. The insulating layers provide structural integrity (e.g., protect various portions of the gas sensor from abrasion and/or vibration, and the like, and provide physical strength to the sensor), and physically separate and electrically isolate various components. The insulating layer(s) can be formed using ceramic tape casting methods or other methods such as plasma spray deposition techniques, screen-printing, stenciling and others. Typically these insulating layers can comprise a dielectric material such as alumina (i.e. aluminum oxide (Al₂O₃), and the like.

In the present exemplary embodiment, insulating layers L2-L3 can be disposed between the heater 16 and the shield 18; insulating layers L4-L7 can be disposed between the optional shield 18 and the temperature sensor 20; insulating layer L8 can be disposed between the temperature sensor 20 and the impedance-measuring device 34; and insulating layer LI can be disposed adjacent to heater 16 opposite insulating layer L8.

Each of the insulating layers L1-L8 can comprise a thickness of about 500 micrometers or so, depending upon the number of layers employed or, more specifically, a thickness of about 50 micrometers to about 200 micrometers. Layer L8 can comprise a thickness sufficient to provide an overall thickness between the impedance-measuring device 34 and the temperature sensor 20 of about 100 micrometers to about 300 micrometers. A thicker insulation layer may be achieved by using thicker green tape or by using multi-layers for the insulation.

In addition, the surface roughness (Rₐ) of the layer L8, disposed adjacent to the impedance-measuring device 34, can be less than or equal to about 0.5 micrometers or, more specifically, less than or equal to about 0.3 micrometers, and even more specifically, less than or equal to about 0.2 micrometers.

Leads (not illustrated) can be disposed across the various insulating layers L1-L8 to enable the electrical connection of external wiring to portions of the heater 16, the impedance-measuring device 34, and the temperature sensor 20. The leads extend from terminal end 10b where they are in electrical communication with various pads (not shown) through corresponding vias (not shown). The leads and vias can comprise an electrically conductive material. The vias, disposed at or near the terminal end 10b of the ammonia-sensing element 10, can comprise holes with electrically conductive material and provide electrical communication through the appropriate layers.

The heater section 12 can comprise a heater 16 (e.g., a ceramic heater), an optional shield 18, a temperature sensor 20, and one or more of the foregoing insulating layers. The heater 16 can comprise a heat-sensing element 22, which may have a serpentine shape, and heater leads 24a, b. Temperature sensor 20 can comprise a temperature-sensing element 26, which may have a serpentine shape, and temperature leads 28a,b.

The heater 16 can be any heater capable of maintaining sensing end 10a at a sufficient temperature to enable the sensing of ammonia. The heater 16 can comprise any material compatible with the operating environment of the ammonia sensor and capable of producing a desired resistance. Suitable materials for the heater include, but are not limited to, platinum (Pt), palladium (Pd), tungsten (W), molybdenum (Mo), and the like, and alloys and combinations comprising at least one of the foregoing. The heater 16 can be disposed onto one of the foregoing insulating layers at a sufficient thickness to attain the desired resistance and heating capability. The heater thickness can be, for example, about 10 micrometers to about 50 micrometers, or so. The heater 16 can be disposed onto one of the insulating layers using various printing techniques such as, for example, thick or thin film printing. Thick film printing techniques are desired for ease of processing and reduced costs in comparison to most thin film techniques.

Optionally, the shield 18 can be disposed between the heater 16 and the temperature sensor 20, and can comprise any material capable of enhancing the electrical isolation of the heater 16 from the temperature sensor 20. The shield 18 isolates electrical influences between the heater 16 and the temperature sensor 20 by dispersing electrical interferences and creating a barrier between a high power source (such as heater 16) and a low power source (such as temperature sensor 20 and/or impedance-measuring device 34). The shield 18 can comprise, for example, a closed layer, a line pattern (connected parallel lines, serpentine, and/or the like), and/or the like. Some possible materials for the shield 18 include, but are not limited to, electrically conductive materials such as metals including platinum (Pt), copper (Cu), silver (Ag), palladium (Pd), gold (Au), and alloys and combinations comprising at least one of the foregoing.

The temperature sensor 20 can be any temperature sensor capable of monitoring the temperature of the sensing end 10a of the ammonia-sensing element 10, such as, for example, a resistance temperature detector (RTD). The temperature-sensing element 26 and the temperature sensor leads 28a,b can comprise any material having a sufficient temperature coefficient of resistance (TCR) to enable temperature determinations, and a melting point sufficient to withstand the processing temperatures of any operation following its formation such as, for example, the calcining temperature of about 1,400° C. (during the co-firing step, as described below). The temperature sensor can comprise, for example, the same materials disclosed above for the heater 16. In the present exemplary embodiment, temperature sensing element 26 can comprise a serpentine shape with a line width of less than or equal to about 0.15 millimeters. The temperature-sensing element can comprise other shapes and sizes.

The sensing section 14 can comprise an impedance-measuring device 34 and a sensing portion 38. The sensing section 14 can be disposed on a side of the temperature sensor 20 opposite the heater 16. The impedance-measuring device 34 can comprise an impedance-measuring element 42 disposed at the sensing end 10a and leads 44a,b extending from the impedance-measuring element 42. The sensing portion 38 can be disposed adjacent to the impedance-measuring element 42 opposite the temperature sensor 20.

If desired, an optional protective divider 36 can be disposed between the sensing portion 38 and the impedance-measuring element 42, and an optional covering 40 can be disposed adjacent to sensing portion 38, opposite the impedance-measuring element 42. Both the protective divider 36 and the covering 40 can be included, if desired.

The impedance-measuring device 34 can be any device that is responsive to changes in the impedance of a sensing material that occur when the material is exposed to a selected gas such as, for example, ammonia contained in a vehicle exhaust gas stream. Accordingly, the impedance-measuring device 34 can comprise various designs capable of measuring impedance changes such as, for example, a capacitor, a two electrode arrangement, a four conductor arrangement, and the like. The impedance-measuring device 34 can be designed such that the impedance (e.g., complex impedance) of the sensing portion 38 or the derived variables, serve as the measured variable.

The impedance-measuring element 42 and leads 44a,b can comprise any material that is electrically conductive, that is not volatile or susceptible to oxidation during subsequent processing steps, or during use, and that does not ion exchange with the ammonia sensing material. Some possible materials that can be used for one or both of the impedance-measuring element 42 and/or leads 44a,b include, but are not limited to, metals, metal alloys, and combinations including at least one of the foregoing. Examples of the foregoing include gold (Au), platinum (Pt), palladium (Pd), gold platinum alloys (Au—Pt), and gold palladium alloys (Au—Pd). Other examples include unalloyed Group VIII refractory metals such as iridium (Ir), osmium (Os), ruthenium (Ru), and rhodium (Rh). Another example is a heavily P/N doped aluminum doped silicon which, in addition to having the foregoing characteristics, forms a hermetic adherent coating that prevents its oxidation.

In the present exemplary embodiment, the impedance-measuring element 42 can comprise a plurality of spaced apart fingers 46 extending from each lead 44a,b in an interdigitated arrangement (such a structure is also known as an interdigitated capacitor (IDC)). In general, increasing the spacing “S” between the centerlines of the fingers 46 and/or decreasing the width “W” of the fingers, increases the sensitivity of the sensor element 10. Thus, the width and spacing of the fingers 46 can be sized and dimensioned, in combination with other features of the sensor element 10, to achieve the desired sensitivity. “Sensitivity” is measured as a comparison of the baseline sensor output (impedance or voltage (V)) in air (i.e. an ammonia concentration of 0 parts per million (ppm)), to the sensor output in air comprising a concentration of 100 ppm ammonia [(V0 ppm-V100 ppm)/V0 ppm)].

The IDC fingers 46 can comprise a width “W” of about 10 micrometers to about 30 micrometers; more specifically about 15 micrometers to about 25 micrometers; and still more specifically about 18 micrometers to about 22 micrometers. The IDC fingers 46 can comprise a spacing “S” of about 15 micrometers to about 50 micrometers; more specifically about 25 micrometers to about 40 micrometers; and still more specifically about 30 micrometers to about 35 micrometers. In an exemplary embodiment, the IDC fingers 46 comprise a width “W” of about 15 micrometers and a spacing “S” of about 25 micrometers. The impedance-measuring element 42 and the leads 44 a, b can be produced in various fashions.

For example, a precursor can be produced using a thick film technique (e.g., a printing technique) to form a layer, or an already pre-structured layer can be employed as a precursor. Subsequent to forming the precursor, it can be fired (e.g., to densify and stabilize the material) and then patterned as desired. The precursor can be fired at temperatures of greater than or equal to about 600° C., e.g., at temperatures of about 800° C. to about 900° C. In practice, a firing time of about 30 minutes at about 850° C. has been found suitable. Patterning can be accomplished, for example, utilizing photolithography. A uniform layer of a photoresist material can be applied over the fired precursor, such as by a spinning method. The photoresist material can comprise a suitable photosensitive resin and a suitable solvent. A photo mask corresponding to the desired structure can then be disposed adjacent to the photoresist and can be illuminated or irradiated by a suitable source such that an area of the photoresist can be removed later by a developer. The area removed is dependent upon the type of photoresist (with a positive photoresist, the irradiated area is removed, and with negative photoresist, the non-irradiated area of the material is removed). Portions of the fired precursor (e.g., gold) can then be etched away from the exposed areas without photoresist, to form the impedance-measuring element e.g. IDC. The residual photoresist then can be removed using a suitable photoresist stripper.

The sensing portion 38 can comprise an ammonia sensing material and an inorganic binder. The ammonia sensing material can comprise any material that is compatible with the operating environment in which the ammonia-sensing element 10 can be used, and that is capable of producing a measurable change in its impedance in response to the presence of ammonia. For example, the ammonia sensing material can comprise a zeolite such as an alumino-silicate with a pentasil crystal structure, in the hydrogen form; an alumino-silicate with a beta crystal structure, in the hydrogen form; an alumino-silicate with a pentasil crystal structure, in the ammonia form; an alumino-silicate with a beta crystal structure, in the ammonia form; and combinations comprising at least one of the foregoing. When the ammonia form of the foregoing zeolites is used, it can be converted to the hydrogen form by a heat treatment, for example, by heating to about 600° C. for a short period of time. The zeolite can comprise a modulus of about 25 to about 400 (i.e. the ratio of silica (i.e. silicon dioxide (SiO₂) to alumina). One possible zeolite is an alumino-silicate pentasil with a modulus of about 80 to about 90. The foregoing ammonia sensing materials have negligible cross-sensitivities to other exhaust species such as HC, CO, NO, and N0₂.

Sensing portion 38 can comprise about 50 wt. % to about 99 wt. % of the ammonia sensing material; more particularly about 75 wt. % to about 95 wt. %; more particularly still about 90 wt. %; based on the total weight of the sensing portion 38. The balance can comprise the inorganic binder, as described in greater detail below.

The ammonia sensing ability of zeolite increases at elevated temperatures, for example, at temperatures of about 350° C. to about 500° C. Therefore, as described above, zeolite based ammonia sensors typically include a heater to heat the sensor to the desired temperature, a temperature sensor to detect the temperature, and an electronic feedback control circuit to provide feedback to the heater in order to regulate the temperature of the sensor. Therefore, it should be understood that materials with sensing abilities that do not change with temperature might not require the use of a heater, temperature sensor and/or feedback circuit.

The inorganic binder can comprise any material that is compatible with the operating environment for which the ammonia-sensing element 10 is designed, and that does not cause a phase change in the ammonia sensing material. The selection of the inorganic binder can be made by one of ordinary skill in the art with routine experimentation based on a variety of factors including, but not limited to, its ability to adhere the particles of ammonia sensing material together and to underlying layers; its tendency to cause a phase change in the ammonia sensing material; its effect on the temperature at which the sensing portion 38 can be calcined; and its effect on sensor sensitivity. The inorganic binder can comprise low impurity levels of Group I, Group II and transition metals, which tend to poison the ammonia sensing material; more specifically, the inorganic binder can comprise individual concentrations of less than about 150 parts per million (ppm) of sodium (Na), potassium (K) and calcium (Ca).

In some embodiments, the inorganic binder can comprise a relatively high surface area material comprising colloidal particles. Some possible materials for the inorganic binder include, but are not limited to, powders of alumina, silica, clay, silicic acid, and combinations comprising at least one of the foregoing.

In other embodiments, the inorganic binder can comprise a first inorganic binder comprising a first surface area, and a second inorganic binder comprising a second surface area different than the first surface area. The first inorganic binder can comprise a surface area of about 0.1 meters-squared per gram (m²/gm) to about 10 m²/gm, and the second inorganic binder can comprise a surface area of about 50 m²/gm to about 500 m²/gm. The first and second inorganic binders can be the same material, or a different material; some possible materials include, but are not limited to, powders of alumina, silica, clay, silicic acid, and combinations comprising at least one of the foregoing.

Sensing portion 38 can comprise about 1.0 wt. % to about 50 wt. %; more particularly about 4.0 wt. % to about 20.0 wt. %; more particularly still about 8.0 wt. % to about 12.0 wt. %; and more particularly still about 10 wt. % of the inorganic binder; based on the total weight of the sensing portion 38. The balance can comprise the ammonia sensing material and other materials, if desired.

In an exemplary embodiment, zeolite can be used as the ammonia sensing material and alumina can be used as the inorganic binder, because the alumina minimizes or prevents phase shifting of the zeolite.

In another exemplary embodiment, the ammonia sensing material can comprise zeolite, and the inorganic binder can comprise CABOSIL HS5, which is an amorphous silica powder with a surface area of about 325 m²/gm and a particle size in the colloidal range (e.g., about 0.005 micrometer to about 0.1 micrometer), and MIN-U-SIL 5, which is a quartz silica powder with a surface area of less than about 5 m²/gm and a particle size of about 5 micrometers.

To form the sensing section 38, a precursor can be formed by forming a mixture of the ammonia sensing material, the inorganic binder(s), an organic binder, and a suitable organic vehicle (e.g., a solvent), followed by printing (e.g., thick film printing). The organic binder provides strength to the green (unfired) layer, and is oxidized during a subsequent firing process. In contrast, the inorganic binder may change in the firing process, but it remains in the fired sensing portion 38. One suitable organic binder is a resin such as ethyl cellulose, which is typically a solid dissolved by the solvent into a liquid. The solvent is added to make a viscous liquid or paste of the mixture. The resulting viscous liquid or paste can be printed into desired patterns at controlled thickness using various thick film printing techniques. Colloidal suspensions of ceramics also may be used, such as colloidal suspensions of alumina and/or silica in water or organic solutes. Alternatively, the inorganic binder can be added as an organometallic such as ethyl-silicone compounds, or metallorganic compounds such as silicon acetate.

The sensing portion 38 can be disposed as a layer over the impedance-measuring element 42. Various techniques can be used to apply the layer such as, for example, the thick film printing techniques as described above. Varying the thickness of the ammonia-sensing portion 38 can affect the adhesion of the ammonia-sensing portion, as well as its sensitivity. For example, increasing the thickness of the layer can reduce the binding ability of the inorganic binder and the adhesion of the layer to any underlying structures. In addition, reducing the thickness of the layer can reduce the sensitivity of the ammonia sensor. Accordingly, it is desirable to form a sufficiently thick sensing portion 38 to provide the desired ammonia sensitivity, without sacrificing the binding or adhesive characteristics of the layer. It should be understood that sensing portion 38 could comprise any thickness that achieves these goals. In some exemplary embodiments, sensing portion 38 can comprise a thickness of greater than or equal to about 40 micrometers; more specifically greater than or equal to about 50 micrometers; more specifically still greater than or equal to about 60 micrometers; and still more specifically greater than about 70 micrometers. In some instances, the thickness of sensing portion 38 can be greater than 70 micrometers provided that the adhesion and sensitivity of the ammonia sensing material are not adversely affected. Thus, the thickness of the sensing section 38 can be selected to achieve the desired sensitivity, in combination with other features of the sensor element 10 such as, for example the finger width and spacing of the IDC.

The optional covering 40 can be disposed adjacent the sensing portion 38, on a side of the impedance-measuring device 34, opposite the temperature sensor 20. The optional covering 40 is designed to protect the leads 44a,b of the impedance-measuring device 34. The covering 40 can comprise any material capable of protecting the leads 44a,b, including, but not limited to, alumina, spinel, glass, and the like, as well as combinations comprising at least one of the foregoing.

The optional protective divider 3636 can be disposed between the impedance-measuring device 34 and the sensing portions 38, and can be designed to provide a barrier to the migration of contaminants in the impedance-measuring device 34 and/or heater section 12, to the sensing portion 38, which would degrade the performance of device 34. The protective divider 36 can comprise a material that has sufficient stability, both morphologically and chemically, to withstand the high temperatures necessary during the service of the sensor. Possible materials for the protective divider 36 comprise dielectric properties that change minimally or not at all, such as silica, alumina, and the like, as well as combinations comprising at least one of the foregoing. The protective divider 36 can comprise a thickness and density sufficient to provide a barrier to contaminants, while allowing an AC electrical signal to pass through. Depending on the material, the protective divider can comprise a thickness of about 50 nanometers to about 500 nanometers. The protective divider 36 can be disposed by various methods including, but not limited to, sol-gel spinning, sputtering, and chemical vapor deposition.

Formation of the sensing element can comprise forming the heater section, disposing the impedance-measuring device on the heater section, optionally disposing the protective divider over the sensing end of the impedance-measuring device, disposing the sensing portion over the protective divider, and disposing the covering over the leads of the impedance-measuring device.

For example, a heater serpentine can be screen printed on to a green insulating layer; while the heater leads can be screen-printed onto the same or an adjacent green insulating layer. These layers can be laid-up such that the heater leads contact the heater serpentine outer legs.

A shield can be screen printed onto a side of the adjacent green layer or onto a third green layer that can be laid-up on a side of the adjacent green layer opposite the heater.

A temperature sensor can be printed onto one or more green layers as with the heater. These layer(s) are laid-up on a side of the shield opposite the heater. Optionally, more green insulating layers can be laid-up between the shield and the temperature sensor. These layers can then be heated to form the heater section e.g., the green layers can be fired to calcine the green layers or alternatively, to allow them to adhere sufficiently such that they can be processed together as a unit in subsequent steps.

An impedance-measuring device (e.g. IDC) can be printed onto the heater section on a side of the temperature sensor opposite the heater. A precursor can be produced using a thick film technique (e.g., a printing technique) to form a layer, or an already pre-structured layer can be employed as a precursor. Subsequent to forming the precursor, it can be fired (e.g., to densify and stabilize the material from which the IDC was formed) and then patterned as desired. The precursor can be fired at temperatures of greater than or equal to about 600° C., e.g., at temperatures of about 800° C. to about 900° C. In practice, a firing time of about 30 minutes at about 850° C. has been found suitable. Patterning can be accomplished, for example, utilizing photolithography. A uniform layer of a photoresist material can be applied over the fired precursor, such as by a spinning method. The photoresist material can comprise a suitable photosensitive resin and a suitable solvent. A photo mask corresponding to the desired structure can then be disposed adjacent to the photoresist and can be illuminated or irradiated by a suitable source such that an area of the photoresist can be removed later by a developer. The area removed is dependent upon the type of photoresist (with a positive photoresist, the irradiated area is removed, and with negative photoresist, the non-irradiated area of the material is removed). Portions of the fired precursor (e.g., gold) can then be etched away from the exposed areas without photoresist, to form the impedance-measuring element e.g. IDC. The residual photoresist then can be removed using a suitable photoresist stripper.

A sensing section can be disposed over the IDC. A precursor can be formed by forming a mixture of the ammonia sensing material, the inorganic binder, a solvent and an organic binder. The precursor then can be printed over the IDC and heated to calcine the sensing portion and form the sensing section. If the green layers of the heater section were not heated to calcination in the earlier heating steps, then the green layers can be calcined simultaneously with the sensing portion.

Optionally, a protective divider can be disposed over at least the sensing end of the impedance-measuring device (e.g., spun on) prior to printing the sensing portion over the sensing end of the impedance-measuring device. Optionally, a covering can be disposed over the leads of the impedance-measuring device to form the ammonia-sensing element. Each of these optional layers can be dried and fired prior to the application of another layer.

To protect the ammonia-sensing element, it can be disposed in a housing to form an ammonia sensor. Although the ammonia sensor can be used in various applications, including factories and the like, it is particularly useful in vehicle exhaust systems, such as, heavy-duty diesel truck applications.

Unless specified otherwise, all dimensions disclosed herein are prior to firing (i.e., in the green state).

The following non-limiting examples further illustrate the various embodiments described herein.

### WORKING EXAMPLES

A comparison of the durability output of two ammonia sensors was made.

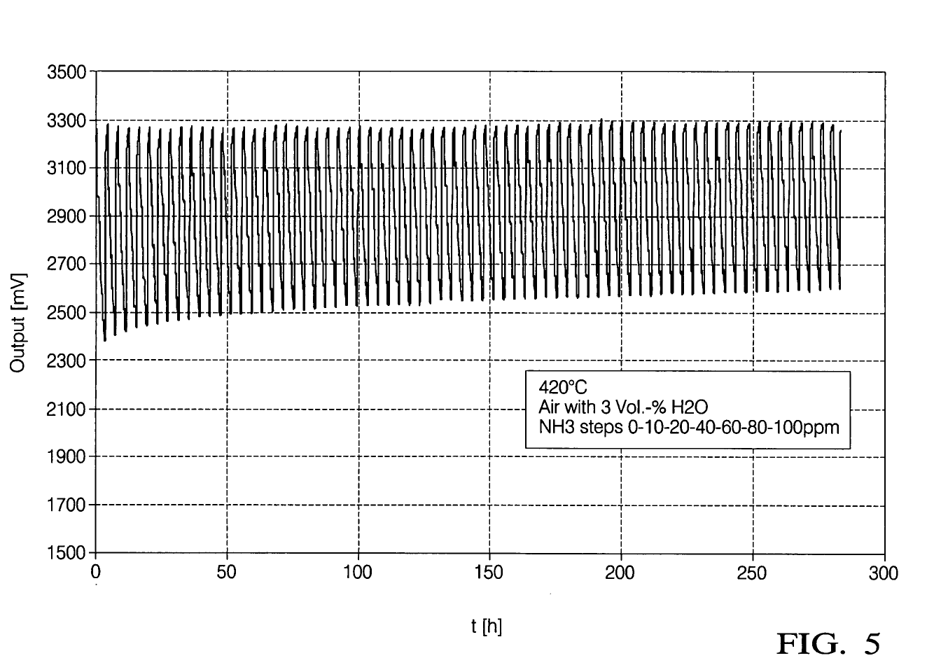

FIG. 5 is a graphical representation of an exemplary ammonia sensor according to the present disclosure. The impedance-measuring device comprised an IDC with a line width “W” of about 15 micrometers and a spacing “S” of about 25 micrometers. The ammonia-sensing portion of the impedance-measuring device comprised zirconia and an alumina binder (i.e. an inorganic binder). The thickness of the ammonia-sensing portion was about 50 micrometers. The sensing element comprised a co-fired heater with varying leg widths (as disclosed in commonly owner and co-pending U.S. patent application Ser. No. 10/909,552, which was filed on Aug. 2, 2004).

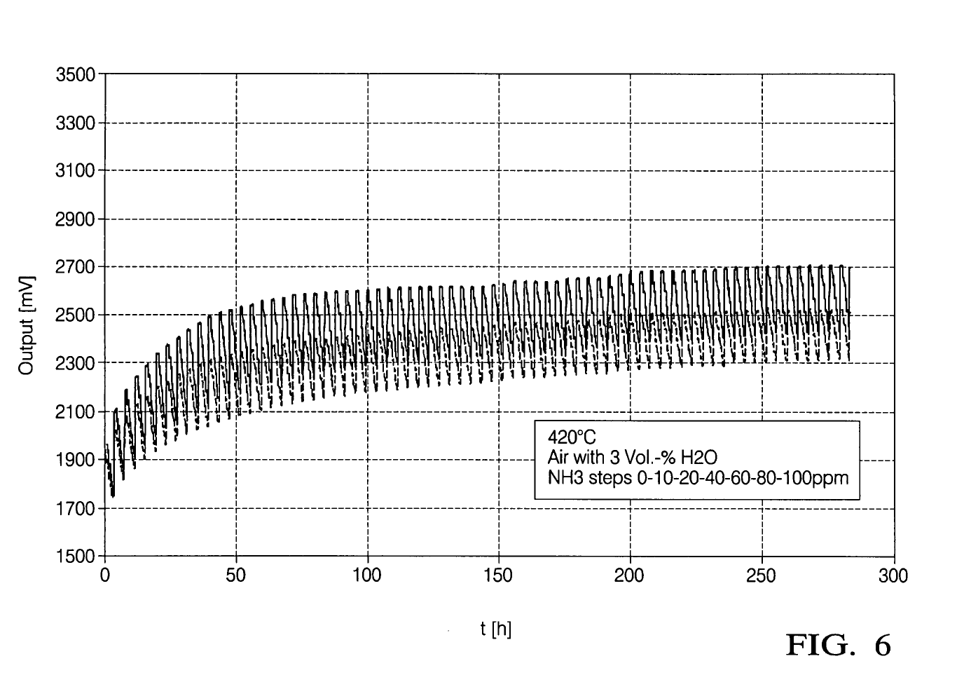

In contrast, the ammonia sensor represented graphically in FIG. 6 comprised an IDC with a line width “W” of about 20 micrometers and a spacing “S” of about 20 micrometers. The ammonia sensing portion comprised zirconia, and did not comprise an inorganic binder. The thickness of the ammonia-sensing portion was about 20 to 30 micrometers. The heater was the same as that of the foregoing sensor.

Comparing the signal amplitude of the graphs of FIGS. 5 and 6 shows that the ammonia sensor represented by FIG. 5 had improved signal sensitivity i.e. about 18-20%, in contrast to the ammonia sensor represented by FIG. 6, which was about 10%.

Also as shown in the graphs, the ammonia sensor represented by FIG. 5 has a stable durability output from the outset (i.e. zero (0) hours) of durability aging, whereas the ammonia sensor represented by FIG. 6 did not have a stable output (amplitude) until about 50 hours of durability aging. Thus, the exemplary ammonia-sensing element provides a stable output in the absence of curing after manufacture, as well as improved sensitivity to ammonia.

In addition, ammonia sensors formed with sensing elements that do not include a binder are prone to poor adhesion to the underlying insulative layer and impedance-measuring element, and substantial variations in the amplitude of the sensor output. The wider spacing of the IDC fingers provides improved manufacturing yields because there is a reduced tendency to have shorts between the fingers after patterning by photolithography/etching, and the thicker ammonia sensing portion improves yields because it results in less impedance variation in the completed sensors.

While the disclosure has been described with reference to an exemplary embodiment, it will be understood by those skilled in the art that various changes can be made and equivalents can be substituted for elements thereof without departing from the scope of the disclosure. In addition, many modifications can be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

What is claimed is:

1. A sensing element, comprising:
a heater section comprising a heater and a temperature sensor;
a sensing section comprising an impedance-measuring device and a sensing portion disposed adjacent the impedance-measuring device opposite the heater section, the sensing portion comprising a mixture of a sensing material and an inorganic binder; and
a first insulating layer disposed between the heater and the temperature sensor and a second insulating layer disposed between the temperature sensor and the impedance-measuring device.

2. The sensing element of claim 1, wherein the sensing material is an ammonia sensing material.

3. The sensing element of claim 2, wherein the ammonia sensing material comprises a zeolite selected from the group consisting of an alumino-silicate with a pentasil crystal structure, in hydrogen form; an alumino-silicate with a pentasil crystal structure, in ammonia form; an alumino-silicate with a beta crystal structure, in the hydrogen form; an alumino-silicate with a beta crystal structure, in the ammonia form; and combinations comprising at least one of the foregoing.

4. The sensing element of claim 3, wherein the zeolite comprises a modulus of about 20 to about 400.

5. The sensing element of claim 1, wherein the inorganic binder is selected from the group consisting of alumina, silica, clay, and combinations comprising at least one of the foregoing.

6. The sensing element of claim 1, wherein the inorganic binder comprises a mixture of a first inorganic binder material and a second inorganic binder material.

7. The sensing element of claim 6, wherein the first inorganic binder material comprises a surface area of about 0.1 m²/gm to about 10 m²/gm and the second inorganic binder material comprises a surface area of about 50 m²/gm to about 500 m²/gm.

8. The sensing element of claim 7, wherein the first inorganic binder material and the second inorganic binder material are selected from the group consisting of alumina, silica, clay, and combinations comprising at least one of the foregoing.

9. The sensing element of claim 1, wherein the sensing portion comprises about 1.0 wt. % to about 50 wt. % of the inorganic binder, based on the total weight of the sensing portion.

10. The sensing element of claim 1, wherein the sensing portion comprises about 50 wt. % to about 99 wt. % of the sending material, based on the total weight of the sensing portion.

11. The sensing element of claim 1, wherein the sensing portion comprises a thickness of about 50 micrometers to about 60 micrometers.

12. The sensing element of claim 1, wherein the impedance-measuring device is an IDC comprising a plurality of interdigitated fingers having a selected width and a selected spacing between the centerlines of the fingers.

13. The sensing element of claim 12, wherein the fingers of the IDC comprise a width of about 10 micrometers to about 30 micrometers.

14. The sensing element of claim 12, wherein the fingers of the IDC comprise a spacing of about 15 micrometers to about 50 micrometers.

15. The sensing element of claim 1, wherein the impedance-measuring device comprises a material selected from the group consisting of metals, metal alloys, and combinations comprising at least one of the foregoing.

16. The sensing element of claim 1, further comprising a third insulating layer disposed between the heater and the temperature sensor, and a shield disposed between the first and third insulating layers.

17. The sensing element of claim 1, wherein the second insulating layer has a surface roughness of less than or equal to about 0.5 micrometers.

18. A method for making a sensing element, comprising:
forming a green laminate heater section comprising a first insulating layer, a heater disposed on one side of the first insulating layer and a temperature sensor disposed on the opposite side of the first insulating layer, and heating the green laminate heater section to form a heater section;
forming an impedance-measuring device pattern on the temperature sensor side of the heater section;
forming a sensing material precursor comprising an inorganic binder and a sensing material;
disposing the sensing material precursor over the impedance-measuring device pattern; and
heating the sensing material pre-cursor to form the sensing element comprising a sensing portion.

19. The method of claim 18, further comprising a shield disposed between the heater and the temperature sensor.

20. The method of claim 18, further comprising forming the impedance-measuring device as an interdigitated capacitor (IDC) comprising a plurality of spaced apart interdigitated fingers

21. The method of claim 20, further comprising forming the fingers of the IDC to have a width of about 10 micrometers to about 30 micrometers and a spacing of about 15 micrometers to about 50 micrometers.

22. The method of claim 18, wherein the sensing portion comprises a thickness of greater than or equal to about 40 micrometers.

23. The method of claim 19, wherein the sensing material comprises an ammonia sensing material.

24. The method of claim 18, wherein the sensing portion comprises about 1.0 wt. % to about 50 wt. % of the inorganic binder, based on the total weight of the sensing portion.

25. The method of claim 18, wherein the sensing portion comprises about 50 wt. % to about 99 wt. % of the sending material, based on the total weight of the sensing portion.
